(19) United States
(12) Patent Application Publication
(10) Pub. No.: US 2009/0149961 A1
(43) Pub. Date: Jun. 11, 2009

(54) Shoulder Prosthesis having a Protrusion on the Base Plate

(75) Inventor: Frank Dallmann, Schmoelln (DE)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: Mathys AG Bettlach, Bettlach (CH)

(21) Appl. No.: 12/300,436

(22) PCT Filed: Apr. 25, 2007
(86) PCT No.: PCT/EP2007/003660

§ 371 Date: Nov. 11, 2008

(30) Foreign Application Priority Data
May 22, 2006 (DE) 10 2006 023 957.1
Sep. 5, 2006 (DE) 10 2006 041 550.7

(51) Int. Cl. A61F 2/40

(52) U.S. Cl. 623/19.11; 623/19.12; 623/19.13

(57) ABSTRACT

A shoulder prosthesis comprising a socket and a condyle that cooperates with the socket. The condyle also cooperates with a base plate facing the scapula similar to a clamping connection, the condyle being plugged onto the base plate by means of a receptacle that is embodied on the condyle and is concentric to the center line thereof. the base plate can be anchored to the glenoid fossa of the scapula with the aid of a protrusion located on the base plate as well as anchoring elements which can be accommodated within sleeves provided on the base plate. According to the invention, the base plate has a dome-shaped bottom surface that is to rest against the glenoid fossa of the scapula, the center of the dome shape being eccentric to the center line of the base plate.

The invention relates to a shoulder prosthesis having a condyle and an articulation socket which co-operates therewith.

A shoulder prosthesis of the type according to the invention is conventionally constructed in such a manner that an articulation socket which co-operates with a condyle is secured to an upper arm bone (humerus) and the condyle is secured in an anatomical glenoid cavity (Cavitas Glenoidalis) of a shoulder blade (scapula). In contrast to the anatomical shoulder joint in which the humerus head of the upper arm bone forms a ball-and-socket joint with the glenoid cavity (Cavitas Glenoidalis) of the shoulder blade, this shoulder prosthesis constitutes an inverse shoulder joint. When the upper arm is moved away from or towards the upper body (abduction/adduction), the articulation socket which is fixed to the humerus rotates about the condyle. If the upper arm is adducted to a comparatively high degree, a portion of the articulation socket protrudes beyond an edge of the condyle in such a manner that an articulation socket edge of the portion abuts a lower edge of the Cavitas Glenoidalis of the shoulder blade (Inferior Notching).

In order to prevent abutment of the articulation socket when the upper arm is adducted to a comparatively high degree, it is already known to mount the condyle in an offset state on the Cavitas Glenoidalis in the direction towards a lower corner (Angulus Inferior) of the shoulder blade. EP 1 607 070 A1 discloses a shoulder prosthesis of this type with an offset condyle. A protrusion which is provided for anchoring on the shoulder blade is arranged centrally relative to the center axis of a base plate at the shoulder blade side on the base plate. Furthermore, the condyle has, for the base plate, a receptacle which is orientated in an eccentric manner relative to the center axis thereof.

The problem addressed by the invention is to provide a shoulder prosthesis with particularly uniform force distribution between the molded portions of the shoulder prosthesis and at anchoring interfaces of the shoulder prosthesis with the anatomical shoulder blade in order to increase the life-span of the material and reliability of the shoulder prosthesis in terms of anchoring. This should also be possible in particular when the glenosphere is positioned in an offset state in an inferior direction in order to reduce inferior notching, with the particularly strong bone content of the central anatomical glenoid cavity being used.

The problem mentioned is solved according to the invention by the features of the independent claims 1 and 5. The dependent claims relate to advantageous developments.

According to a first aspect of the present invention, the shoulder prosthesis comprises an articulation socket and a condyle which co-operates therewith and which co-operates with a scapula-side (shoulder-blade-side) base plate in the manner of a clamping connection. The condyle is fitted to the base plate by means of a receptacle which is formed thereon centrally relative to the center axis thereof and which can be anchored to the Cavitas Glenoidalis (anatomical articulation socket) of the scapula by means of a protrusion which is arranged on the base plate and by means of anchoring elements which can be received in sleeves which are provided on the base plate. According to the invention, there is provision for the base plate to have a dome-like base face which is provided for abutment against the Cavitas Glenoidalis of the scapula, the center of the dome-like shape being arranged so as to be offset in a superior direction eccentrically relative to the center axis of the base plate. Preferably, the protrusion is arranged along the center axis of the dome-like shape of the base face.

According to a second aspect of the present invention, the shoulder prosthesis comprises an articulation socket and a condyle which co-operates with the articulation socket in the manner of a ball-and-socket joint, and a base plate which can be secured at the scapula side (shoulder-blade side) to the Cavitas Glenoidalis (anatomical articulation socket) of the scapula (shoulder blade) by means of a protrusion and anchoring elements and which is connected, at a side facing the articulation socket, to the condyle in the manner of a clamping connection, a receptacle which corresponds to the size of the base plate being formed on the condyle centrally relative to the center axis thereof in order to fit the condyle on the base plate in order to form the clamping connection. Sleeves for receiving the anchoring elements are formed on the base plate and, at the scapula side, the protrusion is formed eccentrically relative to the center axis of the base plate and, in the region of the protrusion, one of the sleeves is formed for securing the base plate to the Cavitas Glenoidalis.

The advantages afforded by the invention are firstly that, by means of the receptacle for receiving the base plate, formed on the condyle centrally relative to the center axis of the condyle, a force which is applied by the condyle to the articulation socket is transmitted in a particularly uniform manner to the base plate and the scapula and an eccentric load of the clamping connection between the base plate and the condyle and material wear, in particular on the ball-and-socket joint and on the anchoring interface between the base plate and the scapula, are prevented. Furthermore, the eccentricity of the base face of the base plate or the protrusion which is arranged thereon eccentrically relative to the center axis of the base plate and one of the anchoring elements which is received in one of the sleeves which is formed in the region of the protrusion on the base plate, provide particularly secure and reliable anchoring of the base plate, which is arranged in an offset manner on the Cavitas Glenoidalis of the scapula. The anchoring element provided according to the second aspect of the invention in the region of the protrusion further increases the reliability of the anchoring of the base plate on the scapula and additionally reduces the load of the protrusion resulting from forces acting on the condyle and the base plate. Furthermore, a material fracture between the base plate and the protrusion and consequently a detachment of the base plate from the scapula are prevented owing to the fact that the anchoring elements absorb forces which act on the protrusion and which are brought about by the articulation socket.

In order effectively to prevent abutment of the articulation socket when the upper arm is adducted to a comparatively large degree towards the upper body, the base plate is configured so as to be secured in an offset manner, preferably on the Cavitas Glenoidalis in the direction towards the Angulus Inferior of the shoulder blade. The center of the dome-like shape of the base face is displaced in the opposite direction to the Angulus Inferior of the scapula and the protrusion is arranged on the base plate at the scapula side at the upper portion of the base plate facing away from the Angulus Inferior. The anchoring of the protrusion on the scapula is reinforced by means of an anchoring element which can be received in a sleeve which is formed in the region of the protrusion on the base plate.

In order reliably to maintain the clamping connection between the condyle and the base plate when the articulation socket is placing a relatively high level of load on the condyle, an outer face of the base plate and a receiving-member-side face which forms the clamping connection therewith are preferably constructed in a circular manner so that a condyle wall between the receiving-member-side face and an outer face of the condyle with which the articulation socket engages has a consistent wall thickness.

In order to prevent twisting of the condyle which co-operates with the base plate by means of the clamping connection on the base plate owing to forces acting radially on the condyle, the condyle has on one portion, preferably at the edge, a groove which co-operates with a tongue which is provided on the base plate.

In order to anchor the base plate in a particularly secure manner, advantageously the tongue is preferably arranged on the base plate on the portion of the base plate facing away from the Angulus Inferior, one of the sleeves for receiving one of the anchoring elements being formed in the region of the tongue on the base plate.

According to an advantageous configuration, the receptacle is arranged on the condyle for receiving the base plate in such a manner that the base plate disappears completely therein and an edge face of the condyle and the base face of the base plate adjoin each other in a substantially flush manner. The scapula-side receptacle of the condyle is thereby closed with respect to the environment and protected against damage caused by external influences.

Since the base plate, in a pre-assembly operation, is first fitted, with the base face of the base plate, on the Cavitas Glenoidalis and anchored in the scapula by means of the protrusion, before the anchoring is carried out by the anchoring elements, it is advantageous to arrange the protrusion on the base plate in a longitudinal direction at right-angles relative to the base face of the base plate.

In order securely to anchor the base plate, the protrusion is preferably configured in the manner of a peg. The protrusion advantageously has ribs which form clamping connections with a hole wall of a hole which is provided for the protrusion in the scapula.

In order to anchor the base plate, there are preferably provided, in addition to the protrusion, the anchoring elements which can be inserted into the sleeves of the base plate. The sleeves constitute passages which are formed on the base plate from one side of the base plate facing away from the scapula to a side of the base plate facing the scapula and, at the side facing away from the scapula, advantageously have recesses in which the anchoring elements can be mounted with heads which are provided thereon.

According to an advantageous configuration, the sleeves are formed on the base plate around the protrusion. The anchoring elements which can be received in the sleeves reinforce the anchoring of the base plate and absorb forces acting on the protrusion in order in particular to increase the reliability of the anchoring and to prevent a material fracture between the protrusion and the base plate.

In an advantageous development for receiving forces that act on the base plate and are brought about by the articulation socket, the anchoring of the base plate by means of the anchoring elements is increased in that at least some of the anchoring elements are fixed in the scapula so as to be spread apart from each other in the manner of the fingers of a wide-open hand. The sleeves which guide these anchoring elements are formed on the base plate accordingly.

According to an advantageous configuration, the anchoring elements are constructed as anchoring screws.

In order to prevent flapping of the base plate on the Cavitas Glenoidalis and to fix the base plate to the scapula in a particularly reliable and secure manner, it is advantageous to form, on the sleeves which are provided for guiding the splayed anchoring elements, screw threads which co-operate with the screw threads of the anchoring elements which are constructed as anchoring screws in order to form angularly stable screw connections. However, the additional sleeves are preferably constructed in such a manner that there is a slight clearance for the substantially parallel orientation of the anchoring elements relative to the protrusion.

In an advantageous development, the base plate comprises, at the condyle side, a hole which extends at the scapula side through the base plate into the protrusion and constitutes, at the condyle side, a continuation of a passage which is formed in the condyle. The passage and the hole are used, for example, to center the condyle when fitting on a base plate by means of a centring pin which is inserted therein or additionally to secure the condyle to the base plate, for example, by means of a clamping workpiece which co-operates with the passage and the hole.

An embodiment of the invention will be explained in greater detail below, with reference to a drawing, in which:

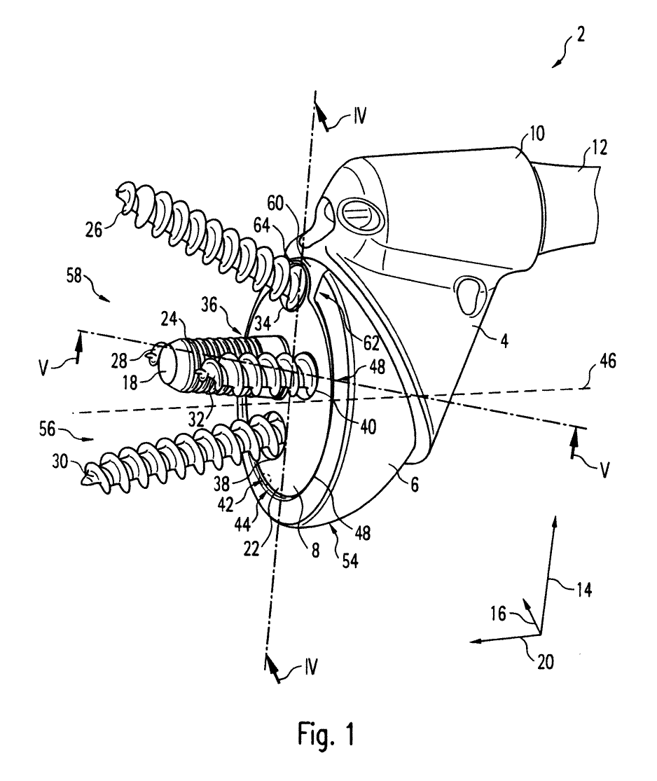

FIG. 1 is a plan view of a shoulder prosthesis with an articulation socket and a condyle and a base plate which is connected thereto with anchoring elements,

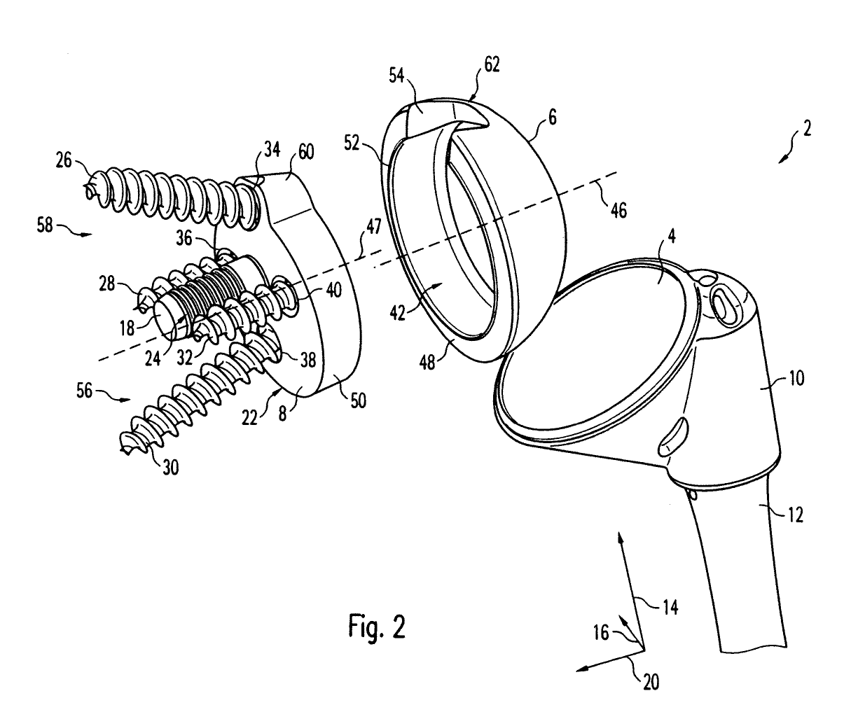

FIG. 2 is an exploded view of the shoulder prosthesis according to FIG. 1,

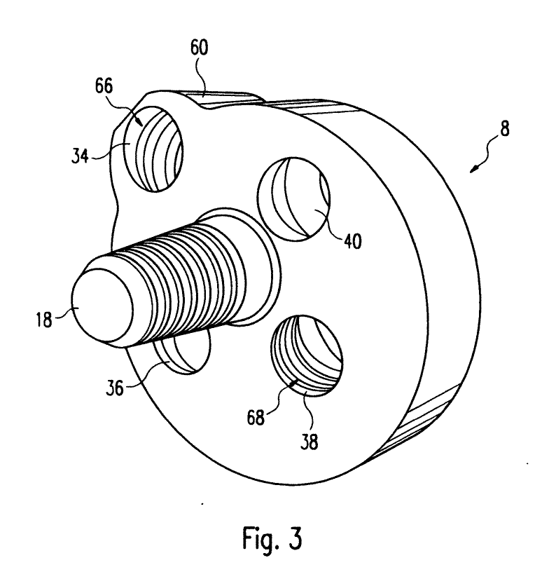

FIG. 3 is a plan view of the base plate according to FIG. 1,

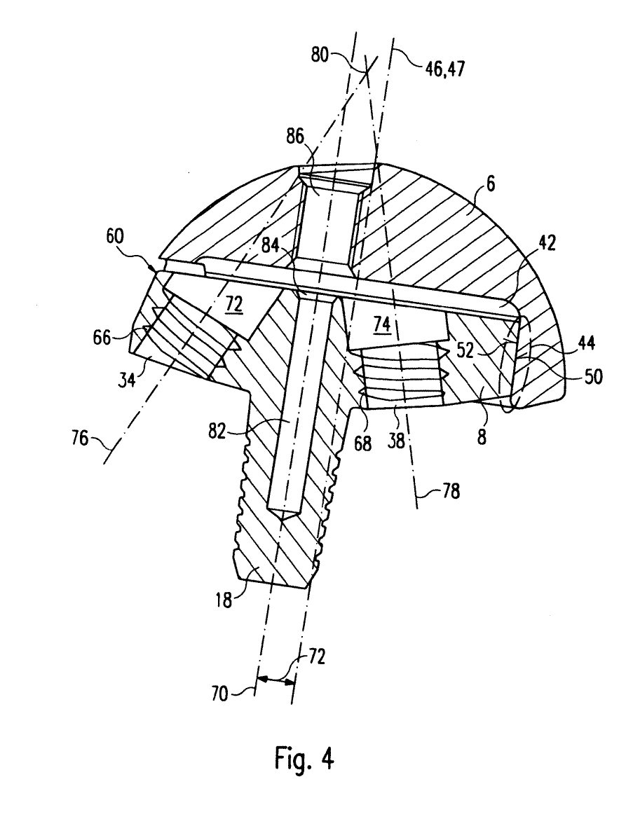

FIG. 4 is a first longitudinal section through the condyle and the base plate which co-operates therewith according to FIG. 1,

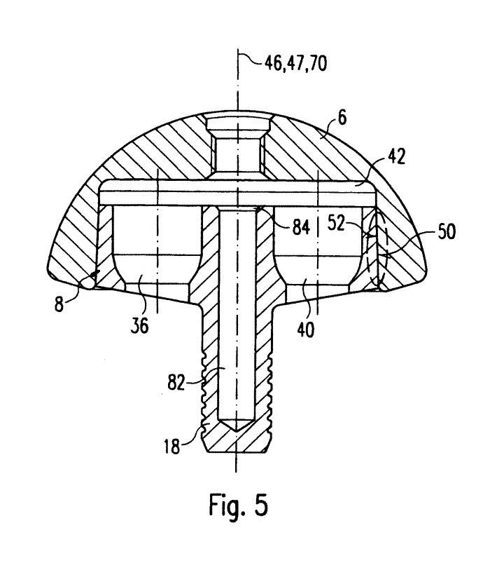

FIG. 5 is a second longitudinal section through the condyle and the base plate according to FIG. 1,

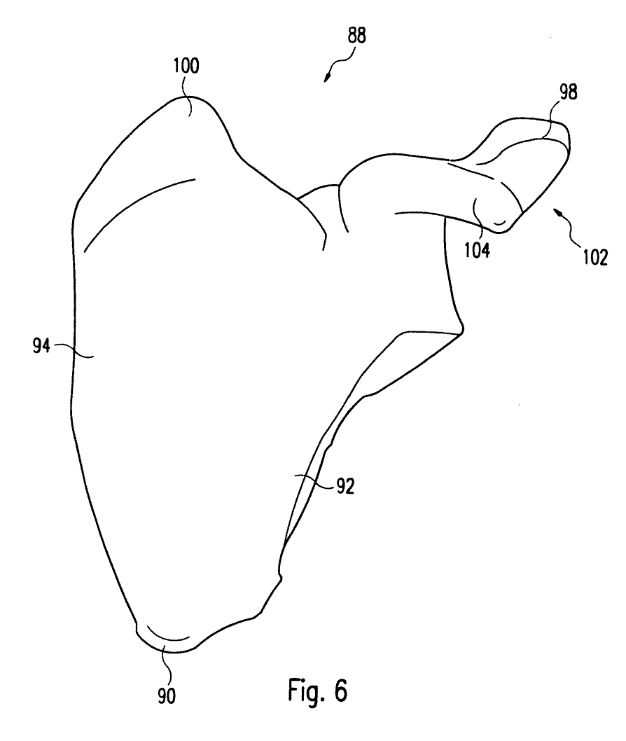

FIG. 6 is a plan view of an anatomical scapula (shoulder blade),

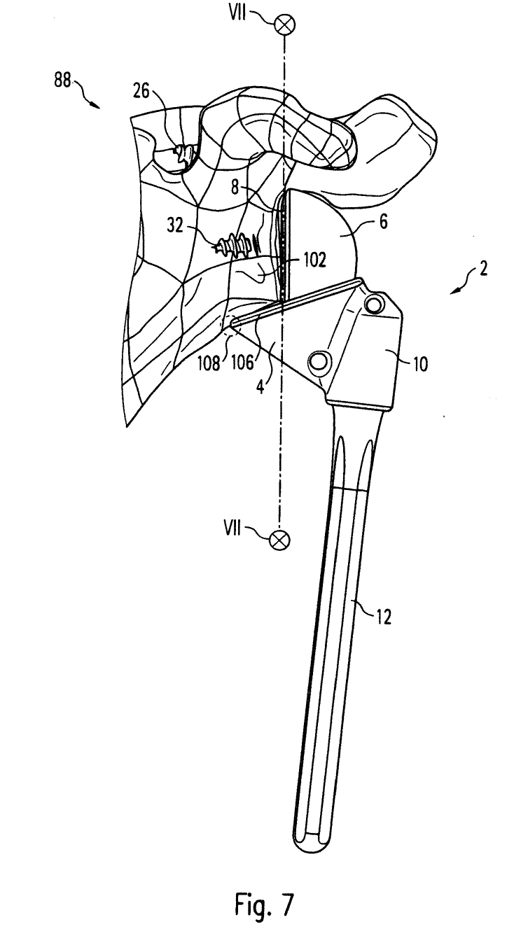

FIG. 7 is a plan view of the shoulder prosthesis according to FIG. 1 mounted on the scapula according to FIG. 6 and

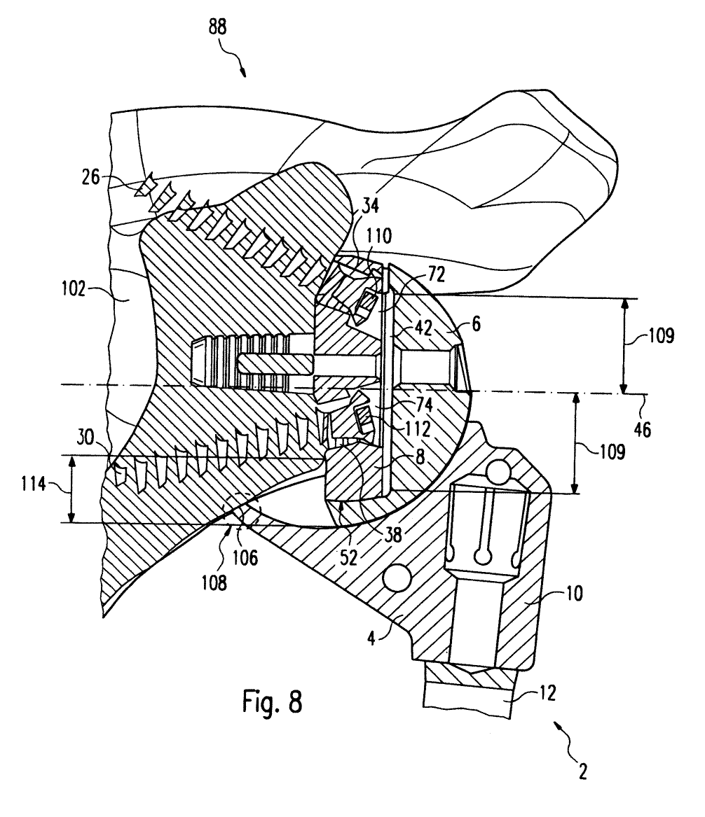

FIG. 8 is a longitudinal section through the shoulder prosthesis mounted on the scapula according to FIG. 7.

Components which correspond to each other are provided with the same reference numerals in all the Figures.

FIGS. 1 and 2 are a plan view and an exploded view of a shoulder prosthesis 2 with an articulation socket 4 which co-operates with a condyle 6 in the manner of a ball-and-socket joint, and a base plate 8 to which the condyle 6 is fitted. The articulation socket 4 is connected by means of a coupling piece 10 to a rod 12 which is recessed in the upper arm (humerus) by means of a humerus shaft which is not illustrated in FIGS. 1 and 2. The articulation socket 4 is provided on the condyle 6 so as to be able to be pivoted in and counter to the directions indicated with the directional arrows 14 and 16. In FIG. 1, the articulation socket 4 is shown in a position in which the upper arm, not illustrated in FIG. 1, is extended away from the body at shoulder or head height.

In order to anchor the shoulder prosthesis 2 to the shoulder blade (scapula) not shown in FIGS. 1 and 2, a protrusion 18 is arranged on the base plate at right-angles relative to a base face 22 of the base plate 8 in an anchoring direction which is indicated with a directional arrow 20. In order to securely anchor the base plate 8, the protrusion 18 is constructed in the manner of a peg and has ribs 24. In addition to the protrusion 18, there are provided anchoring elements 26, 28, 30, 32 which are received in sleeves 34, 36, 38, 40 which are formed on the base plate 8. The anchoring elements 26, 28, 30, 32 are constructed as anchoring screws in the embodiment illustrated. The sleeves 34, 36, 38, 40 which guide the anchoring elements 26, 28, 30, 32 orientate the anchoring elements 26, 28, 30, 32 in such a manner that the upper screw 26 and the lower screw 30 are spread apart from each other in the manner of the fingers of a wide-open hand. The two screws 26, 30, which are guided in an angularly stable manner, bring about securing of the base plate 8 with respect to the bone material of the shoulder blade, whereby even further improved securing of the prosthesis is achieved. The two additional screws 28 and 32 are in contrast primarily used to securely screw the base plate 8 to the shoulder blade initially and to this end are orientated substantially parallel to the protrusion 18. In contrast to the screws 26 and 30, there is a degree of clearance for the orientation of these additional screws 28, 32, whereby the initial securing of the base plate 8 is simplified.

The condyle 6 includes, for receiving the base plate 8, a receptacle 42 whose size is configured to form a clamping connection 44 between the condyle 6 and the base plate 8. The receptacle 42 is formed on the condyle centrally relative to the center axis 46 of the condyle 6. As will be explained in greater detail below, the base face 22 of the base plate 8 has a dome-like shape, the center of which is offset relative to the center axis 47 of the base plate. The protrusion 18 is preferably arranged in the center of this dome-like shape and orientated parallel to the center axis 47 of the base plate. In addition, the receptacle 42 is sized in such a manner that the base plate 8 disappears completely in the receptacle 42 when the condyle 6 is fitted to the base plate 8.

In order to form the clamping connection 44 between the condyle 6 and the base plate 8, an outer face 50 of the base plate 8 co-operates with a receiving-member-side face 52. The outer face 50 and the receiving-member-side face 52 are constructed so as to be circular in such a manner that a condyle wall 54 delimited by the edge face 48 has a consistent wall thickness in the region of the edge face 48. In this context, it should be noted that the connection between the base plate 8 and the condyle 6 could also be produced in a different manner by means of elements which co-operate in a positive-locking and/or non-positive-locking manner. In order to produce the two elements, it is possible to use all materials which are conventionally used in prosthetics, it also being completely possible to use a combination of elements of different materials.

Since the base plate 8 is fixed on the Cavitas Glenoidalis, not illustrated in FIGS. 1 and 2, so as to be offset counter to the arrow direction 14, the base plate 8 at a lower portion 56 cannot be supported completely on the Cavitas Glenoidalis with the base face 22. For this reason, the sleeve 34 in an upper portion 58 for receiving the anchoring element 26 is formed on the base plate 8 in the region of the protrusion 18 which is arranged on the base plate 8 eccentrically relative to the center axis 47 in order to increase the anchoring stability of the base plate 8. In order to form the sleeve 34 on the base plate 8 on the upper portion 58 of the base plate 8 in a manner which is secure in terms of detachment, a protuberance or tongue 60 is arranged on the base plate 8 and provides the necessary space for the sleeve 34. The tongue 60 co-operates with a groove 62 which is provided on the condyle. The tongue-and-groove connection 64 which is thereby formed also serves to fit the condyle 6 correctly to the base plate 8 and prevents the condyle 6 from being twisted on the base plate 8 owing to forces acting radially on the condyle 6.

FIG. 3 is a plan view of the base plate 8 according to FIG. 1 with the protrusion 18 and the sleeves 34, 36, 38, 40, the sleeves 34 and 38 having screw threads 66, 68 which co-operate with screw threads of the anchoring elements (not illustrated in FIG. 3) which are constructed as anchoring screws and which can be received in the sleeves 34, 38 in order to form screw connections. Particularly reliable fixing of the base plate 8 to the Cavitas Glenoidalis can be achieved with the screw connections between the sleeves 34, 38 and the anchoring elements, which are constructed as anchoring screws, and between the anchoring elements and the scapula. By means of the anchoring element that can be received in the sleeve 34, the base plate 8 can be additionally fixed in the region of the tongue 60.

FIGS. 4 and 5 are two longitudinal sections through the condyle 6 and the base plate 8 which co-operates therewith according to FIG. 1. The condyle 6 comprises the receptacle 42 which—as can be seen in particular from the illustration of FIG. 4—is formed thereon centrally relative to the center axis 46 thereof. Furthermore, FIGS. 4 and 5 illustrate the clamping connection 44 between the receptacle side face 52 of the condyle 6 and the outer face 50 of the base plate 8 when the condyle 6 is fitted on the base plate 8.

According to the illustration of FIG. 4, the protrusion 18 is arranged eccentrically relative to the center axis 47 of the base plate 8 which, owing to the centered connection between the condyle 6 and base plate 8, is located precisely on the center axis 46 of the condyle 6, a center axis 70 of the protrusion 18 extending parallel to the center axis 47 of the base plate 8 and being spaced apart from the center axis 47 of the base plate 8 by a spacing 72. In the sectioned view of FIG. 5, all three axes 46, 47 and 70 coincide. It is particularly significant in this instance for the base face 22 of the base plate 8, which abuts the Cavitas Glenoidalis of the shoulder blade when the shoulder prosthesis 2 is in the assembled state, to have a dome-like surface. However, the center axis of this dome does not coincide with the center axis 47 of the base plate 8 but is instead offset relative thereto. To be precise, it forms the axis 70 along which the protrusion 18 is arranged. Because of this specific configuration of the base face 22, there are particular advantages with respect to the assembly of the condyle 6 on the shoulder blade which will be explained in greater detail below.

FIGS. 4 and 5 further illustrate longitudinal sections of the sleeves 34, 38 and 36 and 40 with the screw threads 66, 68 of the first two sleeves 34, 38, the sleeve 34 being formed in the region of the tongue 60 on the base plate 8. In addition to the screw threads, the sleeves have recesses 72, 74 in which the anchoring elements, which are not illustrated in FIGS. 4 and 5, are mounted with heads which are arranged thereon. The sleeves 34 and 38 are formed on the base plate 8 in such a manner that the anchoring elements which can be received in the sleeves 34, 38 spread apart at the scapula side in the manner of two fingers of a wide-open hand. To this end, the sleeves 34, 38 are constructed, with respect to the center axis 47 of the base plate 8, in an angled manner in the longitudinal direction on the base plate 8 such that center axes 76, 78 of the sleeves 34, 38 intersect in continuation at the condyle side. In FIG. 4, an intersection point 80 formed by the center axes 76, 78 of the sleeves 34, 38 is indicated.

In contrast to the sleeves 34 and 38 for the screws 26 and 30 which are mounted in an angularly stable manner, the two additional sleeves 36 and 40 are configured according to the illustration in FIG. 5 in such a manner that the associated screws are orientated in a substantially parallel manner relative to the protrusion 18. The screws 28 and 32 which are guided in these sleeves 36 and 40 first serve to screw the base plate 8 securely to the shoulder blade before the splayed connection is brought about using the two screws 26 and 32. The sleeves 36 and 40 therefore have no thread and are further constructed in the manner of a spherical portion in their head region. If screws with a spherical head are used as anchoring elements, they can be pivoted within a small angular range. This slight clearance allows the screws to be introduced into the shoulder blade in a more simple manner.

In addition to the sleeves 34, 36, 38 and 40, a bore 82 is provided in the base plate 8 and has, at the condyle side, an open end 84 and extends into the protrusion 18 at the scapula side. In continuation of the hole 82, a passage 86 is formed in the condyle 6. The passage 86 and the hole 82 serve, for example, to receive a centring pin which is not illustrated in FIGS. 4 and 5 in order to center the condyle 6 when being fitted to the base plate 8 or to receive a clamping workpiece which is not illustrated in FIGS. 4 and 5 and which co-operates with the passage 86 and the hole 82 in order to additionally fix the condyle 6 to the base plate 8.

FIG. 6 is a plan view of an anatomical scapula 88 (shoulder blade) whose basic form is triangular. The lower corner of the triangle is referred to as the Angulus Inferior 90, which a right-hand edge “Margo Lateralis 92” and a left-hand edge “Margo Medialis 94” adjoin. The Margo Lateralis 92 and the Margo Medialis 94 delimit the upper edge “Margo Superior 96” at a right-hand corner “Angulus Lateralis 98” and a left-hand corner “Angulus Superior 100”. At the Angulus Lateralis 98, an anatomical articulation socket “Cavitas Glenoidalis 102” is positioned and has a coracoid process “Processus Coracoideus 104” which is curved forwards and outwards.

FIG. 7 is a plan view of the shoulder prosthesis 2 according to FIG. 1 which is mounted on the scapula 88 according to FIG. 6 and which has a condyle 6 and a base plate 8. The articulation socket 4 which is secured to the rod 12 by means of the coupling piece 10 is fixed to the upper arm bone (humerus) which is not illustrated in FIG. 7. The base plate 8 is anchored to the Cavitas Glenoidalis 102 of the scapula 88 by means of the protrusion which is not illustrated in FIG. 7 and anchoring elements. FIG. 7 illustrates only ends of the anchoring elements 26 and 32 which are constructed as anchoring screws.

FIG. 7 illustrates the articulation socket 4 in the position of the upper arm adducted towards the upper body, in which the base plate 8, which is arranged in an offset manner on the Cavitas Glenoidalis 102 in the direction of the Angulus Inferior 90, prevents abutment of an articulation socket edge 106 when the upper arm is adducted to a comparatively large extent.

FIG. 7 illustrates, at the lower edge of the Cavitas Glenoidalis 102, which adjoins the Margo Lateralis 92 of the scapula 88, a contact location 108 against which the articulation socket edge 106 abuts if the upper arm is adducted to a non-relatively high degree.

FIG. 8 is a longitudinal section through the shoulder prosthesis 2 which is mounted on the scapula 88 according to FIG. 7 with the condyle 6 and the base plate 8 and the articulation socket 4 which is retained on the humerus (not illustrated in FIG. 8) by means of the coupling piece 10 and the rod 12. The condyle 6 is fitted to the base plate 8 with the receptacle 42. The receptacle 42 is centrally formed on the condyle around the center axis 46 of the condyle 6 and laterally delimited by a receptacle side face 52 which extends in a circular manner around the center axis 46, in this configuration with a radius 109.

The base plate 8 is anchored to the Cavitas Glenoidalis 102 with the protrusion 18 and the anchoring elements. FIG. 8 shows only the anchoring elements 26 and 30 which are constructed as anchoring screws and which are received at the condyle side in the sleeves 34, 38 of the base plate 8 and are mounted in the recesses 72, 74 of the sleeves 34, 38 with heads 110, 112 arranged on the anchoring elements 26, 30. The sleeve 34 is formed in the region of the tongue 62 on the base plate 8 in order to fix the base plate 8, at the side facing away from the Angulus Inferior (not shown in FIG. 8), additionally to the Cavitas Glenoidalis 102 of the scapula 88.

As illustrated in FIG. 8, the base plate 8 is arranged offset in the direction of the Angulus Inferior 90 by a distance 114, whereby abutment of the articulation socket edge 106 is prevented when the upper arm is adducted towards the upper body. Only when the upper arm is adducted to a relatively high degree is abutment of the articulation socket edge 106 at the contact location 108 of the Cavitas Glenoidalis 102 not prevented.

This offset arrangement of the base plate 8 is particularly promoted by the fact that—as explained above—the center of the dome shape of the base face 22 of the base plate 8 is arranged eccentrically relative to the center thereof. In particular, this center is displaced in the opposite direction to the Angulus Inferior 90 of the scapula 88. This specific configuration also results in the surface of the Cavitas Glenoidalis 102 being able to have a spherical-portion-shaped recess which is advantageous for assembly of the shoulder prosthesis 2 since the center of the support face of the base face 22 coincides with the center of the dome-like shape. The processing of the Cavitas Glenoidalis 102 required prior to assembly is therefore particularly simple for a surgeon in comparison with the previously known solution, it still being possible to achieve complete abutment of the base face 22 on the Cavitas Glenoidalis 102 over the entire contact face. It is significant in this instance that the shape of the base plate 8 is optimally adapted to the specific bone conditions, whereby simple but also particularly reliable securing of the prosthesis on the shoulder blade is made possible. This constitutes a significant advantage in comparison with solutions from the prior art in which the bone must be heavily processed or manipulated in order to allow the prosthesis to be secured.

Since the protrusion 18 is further arranged along the longitudinal axis 70 of the dome shape of the base face 22 and consequently in the center of the contact face between the base face 22 and Cavitas Glenoidalis 102, there can be ensured central and consequently optimal force transmission which allows reliable anchoring of the condyle 6 on the scapula 88. In particular shearing forces are particularly well taken up in this instance without the danger of loosening. The solution according to the invention therefore allows the condyle 6 to be mounted on the shoulder blade 2 in a manner which is not only simple to carry out but also particularly reliable.

The invention is not limited to the embodiment illustrated in the drawing. All of the features described above and illustrated in the drawing can be combined with each other in any desired manner.

1.-18. (canceled)

19. A shoulder prosthesis comprising:
an articulation socket;
a scapula-side base plate, said base plate having a receptacle formed thereon centrally with respect to a center axis of the base plate, a protrusion arranged on the base plate, and sleeves provided on the base plate;
a condyle which co-operates with the articulation socket and with the scapula-side base plate in the manner of a clamping connection, the condyle being fitted to the base plate by the receptacle and which can be anchored to the Cavitas Glenoidalis of the scapula by the protrusion and anchoring elements which can be received in the sleeves provided on the base plate;
wherein the base plate has a dome-like base face which is provided for abutment against the Cavitas Glenoidalis of the scapula, the center of the dome-like shape being arranged eccentrically relative to the center axis of the base plate.

20. A shoulder prosthesis according to claim 19, wherein the protrusion is arranged along a center axis of the dome-like shape of the base face.

21. A shoulder prosthesis according to claim 20, wherein one of the sleeves for receiving an anchoring element is formed in the region of the protrusion on the base plate.

22. A shoulder prosthesis according to claim 19, wherein the center of the dome-like shape of the base face is displaced in a direction opposite to an Angulus Inferior of the scapula.

23. A shoulder prosthesis according to claim 19, wherein a receptacle side face on the condyle and an outer face of the base plate that co-operates therewith are constructed in a circular manner to form the clamping connection.

24. A shoulder prosthesis according to claim 19, wherein at a side facing away from the Angulus Inferior of the scapula, a groove is provided on the condyle, with which the base plate co-operates by a tongue formed on the base plate.

25. A shoulder prosthesis according to claim 24, comprising a sleeve formed in the region of the tongue on the base plate.

26. A shoulder prosthesis according to claim 25, wherein the protrusion is arranged on the base plate in a longitudinal axis at right-angles to the base face of the base plate.

27. A shoulder prosthesis according to claim 19, wherein the protrusion has peg-like ribs.

28. A shoulder prosthesis according to claim 19, wherein the sleeves, at a side of the base plate facing away from the scapula, have recesses for heads which are provided on the anchoring elements.

29. A shoulder prosthesis according to claim 19, wherein the sleeves are formed on the base plate around the protrusion.

30. A shoulder prosthesis according to claim 19, wherein at least some of the sleeves on the base plate for guiding the anchoring elements are constructed in such a manner that associated anchoring elements are spread apart from each other in the manner of fingers of a wide-open hand when the shoulder prosthesis is in the final assembled state.

31. A shoulder prosthesis according to claim 30, comprising additional sleeves on the base plate for guiding the anchoring elements constructed in such a manner that associated anchoring elements are oriented with a slight clearance substantially parallel to the protrusion.

32. A shoulder prosthesis according to claim 19, wherein the anchoring elements are anchoring screws.

33. A shoulder prosthesis according to claim 32, comprising screw threads provided on the sleeves for receiving splayed anchoring screws to form screw connections between the base plate and the anchoring screws.

34. A shoulder prosthesis according to claim 19, comprising a bore formed on the base plate in the region of the protrusion in continuation of a passage provided
